(19) United States
(12) Patent Application Publication
(10) Pub. No.: US 2004/0185020 A1
(43) Pub. Date: Sep. 23, 2004

(54) Cosmetic compositions comprising at least one amphoteric surfactant and at least one silicone and their uses

(76) Inventors: Jonathan Gawtrey, Boulogne (FR); Mireille Maubru, Chatou (FR); Nathalie Pinel, Neydens (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/739,100

(22) Filed: Dec. 19, 2003

Related U.S. Application Data
(60) Provisional application No. 60/439,466, filed on Jan. 13, 2003.

(30) Foreign Application Priority Data
Dec. 19, 2002 (FR) 02 216192

(51) Int. Cl. A61K 7/06; A61K 7/11

(52) U.S. Cl. 424/70.12; 554/46

(57) ABSTRACT

A novel detergent cosmetic compositions comprising, in a cosmetically acceptable medium, at least one amphoteric surfactant chosen from alkylamphohydroxyalkylsulphonate compounds and salts thereof and at least one silicone; these compositions provide at least one cosmetic property (such as, smoothness, lightness, softness) without the phenomenon of regreasing the keratinous fibres; as well as the uses of these compositions, for example, for washing and/or conditioning a keratinous substance, such as the hair or the skin.

This application claims benefit of U.S. Provisional Application No. 60/439,466, filed Jan. 13, 2003.

Disclosed herein are novel cosmetic compositions comprising, in a cosmetically acceptable medium, at least one amphoteric surfactant chosen from alkylamphohydroxyalkylsulphonate compounds and salts thereof and at least one specific silicone.

Detergent compositions, such as shampoos and shower gels, based essentially on conventional surface-active agents, for example, anionic, non-ionic and/or amphoteric surface-active agents, further, for example, anionic surface-active agents, are commonly used for cleaning and/or washing hair and/or skin. These compositions can be applied to wet hair or skin and the foam generated by massaging or rubbing with the hands may make it possible, after rinsing with water, to remove the various types of dirt initially present on the hair or skin.

These base compositions may have a good washing power but the intrinsic cosmetic properties associated with these compositions may remain fairly weak. For example, because of this relatively aggressive nature such a cleaning treatment can, in the long term, result in more or less marked damage to a keratinous substance, related, for example, to the gradual removal of the lipids or proteins present in or at the surface of the keratin substance.

Consequently, in order to improve at least one of the cosmetic properties of the above detergent compositions and, for example, of those which may be called upon to be applied to sensitized hair (i.e. hair which is found to be damaged or embrittled, for example, under the chemical action of atmospheric agents and/or of hair treatments, such as permings, dyeings or bleachings), it is now common to introduce into the cosmetic composition at least one additional cosmetic agent. Known as conditioning agents, they are often intended to repair or limit at least one of the harmful or undesirable effects brought about by the various treatments or attacks to which the hair fibres may be repeatedly subjected. These conditioning agents may, of course, also improve the cosmetic behaviour of natural hair.

Currently, the most widely used conditioning agents in shampoos are cationic polymers, silicones and/or silicone derivatives. These agents may confer, on dry or wet washed hair, at least one of the following properties: ease of disentangling, softness and smoothness. As used herein these properties are defined with respect to that which can be obtained with the corresponding cleaning compositions which are devoid of these agents.

However, in the case of sensitized hair, hair washed with a shampoo comprising silicones can be stiff, that is to say lacking suppleness or manageability.

Thus, and in spite of the progress achieved recently in the field of shampoos based on silicone and/or cationic polymers, these compositions may not be completely satisfactory. In some areas, a need currently exists for products exhibiting a better performance with regard to at least one of the cosmetic properties mentioned above.

Alkylamphohydroxyalkylsulphonate amphoteric surfactants have already been recommended in detergent cosmetic compositions. These surfactants have been disclosed, for example, in Patent Application No. WO. 99/36054. Hair washing compositions, however, using these surfactants alone may not result in satisfactory cosmetic properties.

Disclosed herein are thus cosmetic compositions comprising, in a cosmetically acceptable medium, at least one amphoteric surfactant chosen from alkylamphohydroxyalkylsulphonate compounds and salts thereof and at least one silicone chosen from non-organomodified silicones with a viscosity ranging from 500 mm²/s (500 centistokes) to 1 000 000 centistokes) and organomodified silicones, exhibiting at least one cosmetic property, such as, hair disentangling, smoothness, suppleness, manageability and softness.

These novel compositions may provide better deposition of these silicones on a keratinous substance, such as on hair, than a composition comprising conventional amphoteric surfactants, such as cocoamphodiacetate salts.

In another embodiment, the compositions disclosed herein may confer on a keratinous substance, such as hair, at least one notable treating effect, such as, ease of disentangling, lightness, smoothness, softness, manageability and suppleness. This may, for example, be provided without any sensation of a feeling of heaviness.

Further disclosed herein is a method of manufacturing a cosmetic composition comprising including in the composition at least one amphoteric surfactant chosen from alkylamphohydroxyalkylsulphonate compounds and salts thereof and at least one silicone chosen from non-organomodified silicones with a viscosity ranging from 500 mm²/s (500 centistokes) to 1 000 000 mm²/s (1 000 000 centistokes) and organomodified silicones.

Further disclosed herein is a process for the treatment of a keratinous substance, such as hair, comprising applying, to the keratinous substance, the cosmetic compositions disclosed herein.

Even further disclosed herein is the use of the disclosed compositions for providing at least one of the following to hair: disentangling, smoothness, volume, lightness, softness, suppleness and manageability.

As used herein, the term “keratinous substance” means hair, eyelashes, eyebrows, skin, nails, mucous membranes or scalp and, for example, hair.

In one embodiment, the at least one amphoteric surfactant may be chosen from alkylamphohydroxyalkylsulphonate compounds and salts thereof of the following formula (I):

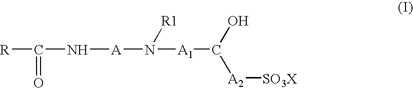

wherein:

R is chosen from saturated and unsaturated and linear and branched hydrocarbonaceous radicals comprising from 5 to 29 carbon atoms;

R may, for example, be chosen from alkyl radicals and mono- and polyunsaturated alkenyl radicals comprising, for example, from 5 to 29 carbon atoms, further, for example, from 7 to 22 carbon atoms and, even further, for example, from 9 to 17 carbon atoms;

R1 is chosen from C₁-C₄ hydroxyalkyl radicals, for example, a hydroxyethyl radical;

A, A₁ and A₂, which may be identical or different, are each chosen from linear and branched, divalent, C₁-C₁₀ radicals, such as C₁-C₁₀ alkylene radicals, for example, C₁-C₃ alkylene radicals; and

X is chosen from a hydrogen atom and inorganic and organic cations, such as,

alkali metals (for example Na⁺ and K⁺), alkaline earth metals (Mg²⁺, Ca²⁺), a NH₄⁺ ion, and ammonium ions resulting from basic amino acids and from aminoalcohols.

In one embodiment, the compounds of formula (I) are chosen from compounds wherein R is chosen from saturated, linear and branched, alkyl radicals comprising from 7 to 29 carbon atoms, for example, from 7 to 22 carbon atoms.

When X is chosen from ammonium ions resulting from an alkanolamine, the alkanolamine can be chosen from monoethanolamine, diethanolamine, triethanolamine and 3-amino-1,2-propanediol. When X is chosen from ammonium ions resulting from an amine, the amine can be chosen from basic amino acids, such as lysine, arginine, sarcosine, ornithine and citrulline.

For example, in formula (I), A and A₂ may each be a —CH₂CH₂— radical.

Further, for example, A₁ may be a —CH₂— radical.

Even further, for example, X may be a Na⁺ ion.

For example, the at least one amphoteric surfactant of formula (I) may be chosen from salts of cocoylamphohydroxypropylsulphonate, for example, the sodium salt, such as the product sold under the name MIRANOL CSE by Rhodia Chimie, and salts of palmitoylamphohydroxypropylsulphonate, for example, the sodium salt, such as the product sold under the name MIRANOL CS by Rhodia Chimie.

In one embodiment, the at least one amphoteric surfactant may be present in the composition disclosed herein in an amount ranging, for example, from 0.1% to 30% by weight, further, for example, from 1% to 20% by weight, further, for example, from 1.5% to 15% by weight, and, even further, for example, from 2% to 8% by weight, relative to the total weight of the composition.

The at least one silicone may, for example, be soluble or insoluble in water. The at least one silicone may also, for example, be soluble or insoluble in the final composition. For example, the compositions disclosed herein can comprise at least one silicone which is insoluble in the composition.

The insoluble silicones may, for example, be dispersed in the compositions disclosed herein in the form of particles having a number-average size, for example, ranging from 2 nanometers to 100 microns, further, for example, ranging from 20 nanometers to 20 microns. The number-average size of the insoluble silicones are measured with a particle sizer.

As used herein, the term “silicone” or “polysiloxane” is understood to mean, in accordance with what is generally accepted by one skilled in the art, any organosilicone polymer or oligomer with a branched, crosslinked, linear or cyclic structure of variable molecular weight obtained by polymerization and/or polycondensation of suitably functionalized silanes and essentially comprising a repetition of main units wherein the silicon atoms are connected to one another via at least one entity chosen from oxygen atoms (siloxane bond ≡Si—O—Si≡), hydroxyl radicals and optionally substituted hydrocarbonaceous radicals being directly bonded via a carbon atom to the silicon atoms. Examples of hydrocarbonaceous radicals include alkyl radicals, for example, C₁-C₁₀ alkyl radicals, such as methyl radicals, fluoroalkyl radicals, aryl radicals, such as phenyl radicals, and alkenyl radicals, such as vinyl radicals (non-organomodified silicone). Other types of radicals capable of being bonded either directly or via a hydrocarbonaceous radical to the siloxane chain include, for example, hydrogen; halogens; such as chlorine, bromine or fluorine; thiols; alkoxy radicals; polyoxyalkylene (or polyether) radicals; and, for example, polyoxyethylene and/or polyoxypropylene radicals; hydroxyalkyl radicals; amide groups, acyloxy or acyloxyalkyl radicals; and anionic groups, such as carboxylates, thioglycolates, sulphosuccinates, thiosulphates, phosphates and sulphates. This list of examples, of course, is in no way limiting.

As used herein, the term “non-organomodified silicone” means, a polysiloxane comprising, on the silicon atoms, at least one group chosen from hydroxyl groups, alkyl groups, alkenyl (for example, vinyl) groups, and aryl groups, wherein these groups are unsubstituted.

In one embodiment, the non-organomodified silicones disclosed herein may have, for example, a viscosity ranging from 500 mm²/s (500 centistokes) to 1 000 000 mm²/s (1 000 000 centistokes). The non-organomodified silicones may, for example, be chosen from polysiloxanes which are insoluble in the composition disclosed herein. In one embodiment, these silicones can be provided in a form chosen from oils, waxes, resins and gums.

The non-organomodified silicones may, further, for example, have a viscosity ranging from 5 000 to 750 000 mm²/s at 25° C.

The viscosity of these silicones, as used herein, is measured at 25° C. according to Standard ASTM 445, Appendix C.

As used herein, silicones which are insoluble in water refer to silicones that are insoluble in water at a concentration of greater than or equal to 0.1% by weight in water at 25° C., that is to say that they do not form, to the naked eye, a transparent isotropic solution.

The organopolysiloxanes are defined in more detail in the work by Walter Noll, “Chemistry and Technology of Silicones”, (1968), Academic Press.

In one embodiment, the non-organomodified silicones may be chosen from nonvolatile silicones and, further, for example, from at least one of the following:

(i) polydialkylsiloxanes;

(ii) polydiarylsiloxanes;

(iii) polyalkylarylsiloxanes;

(iv) silicone gums; and

(v) silicone resins.

The alkyl radicals may comprise, for example, from 1 to 10 carbon atoms and may, further, for example, be a methyl radical. The aryl radicals may, for example, be a phenyl radical.

The polydialkylsiloxanes may, for example, be chosen from polydimethylsiloxanes comprising trimethylsilyl or dimethylsilanol end groups, including:

linear polydimethylsiloxanes comprising trimethylsilyl end groups and having a viscosity ranging from 5 000 to 750 000 mm²/s at 25° C., for example, and without implied limitation, the SILBIONE oils of the 70047 series sold by Rhodia Chimie, further, for example, SILBIONE 70047 V 500 000 oil from Rhodia Chimie, and the polydimethylsiloxane with a viscosity of 300 000 cSt sold under the name DC 200 Fluid 300 000 by Dow Corning, and the oils of the 200 series from Dow Corning, such as DC 200 with a viscosity of 60 000 cSt;

linear polydimethylsiloxanes comprising hydroxydimethylsilyl end groups (dimethiconol according to the CTFA nomenclature), such as the oils of the 48 V series from Rhodia Chimie.

A further example includes polyalkylsiloxanes sold by Goldschmidt under the trade names ABILWAX 9800 and ABILWAX 9801, which are poly(C₁-C₂₀)alkylsiloxanes.

The polyalkylarylsiloxanes may, for example, be chosen from linear and branched polydimethylmethylphenylsiloxanes and polydimethyldiphenylsiloxanes with a viscosity ranging from 500 to 1 000 000 mm²/s at 25° C.

For example, the polyalkylarylsiloxanes may be chosen from the following products sold under the following names:

the SILBIONE oils of the 70 641 series from Rhône-Poulenc;

the oils of the RHODORSII 70 633 and 763 series from Rhône-Poulenc;

the oil Dow Corning 556 Cosmetic Grade Fluid from Dow Corning;

the silicones of the PN and PH series from Bayer, such as the products PN1000 and PH1000; and

certain oils of the SF series from General Electric, such as SF 1023, SF1154, SF1250 and SF 1265.

The silicone gums may, for example, be chosen from polydiorganosiloxanes with a number-average molecular mass ranging from 200,000 to 1,000,000, wherein the polyorganodisiloxanes are used alone or as a mixture in at least one solvent. The at least one solvent may, for example, be chosen from volatile silicones, polydimethylsiloxane (PDMS) oils, polyphenylmethylsiloxane (PPMS) oils, isoparaffins, polyisobutylenes, methylene chloride, pentane, dodecane, and tridecanes.

Examples of silicone gums include:

polydimethylsiloxane,

poly[(dimethylsiloxane)/(methylvinylsiloxane)],

poly[(dimethylsiloxane)/(d iphenylsiloxane)],

poly[(dimethylsiloxane)/(phenylmethylsiloxane)], and

poly[(dimethylsiloxane)/(diphenylsiloxane)/(methylvinylsiloxane)].

Further, for example, the products which can be used in the compositions disclosed herein can be chosen from mixtures such as:

mixtures formed from a polydimethylsiloxane hydroxylated at the chain end (named dimethiconol according to the nomenclature of the CTFA dictionary) and from a cyclic polydimethylsiloxane (named cyclomethicone according to the nomenclature of the CTFA dictionary), such as the product Q2 1401 sold by Dow Corning;

mixtures formed from a polydimethylsiloxane gum with a cyclic silicone, such as the product SF 1214 Silicone Fluid from General Electric; this product is a gum SF 30, corresponding to a dimethicone having a number-average molecular weight of 500 000, dissolved in the oil SF 1202 Silicone Fluid, corresponding to decamethylcyclopentasiloxane; and

mixtures of two PDMSs with different viscosities, for example, of a PDMS gum and of a PDMS oil, such as the product SF. 1236 from General Electric.

The organopolysiloxane resins which can be used in the composition disclosed herein are crosslinked siloxane systems comprising at least one unit chosen from:

(R)₂SiO_{2/2}, (R)₃SiO_{1/2}, RSiO_{3/2} and SiO_{4/2}, wherein R is chosen from hydrocarbonaceous groups comprising from 1 to 16 carbon atoms and a phenyl group. These resins may, further for example, be chosen from those resins wherein R is chosen from lower C₁-C₄ alkyl radicals, such as a methyl radical, and a phenyl radical.

Mention may be made, among these resins, of the product sold under the name “Dow Corning 593” or the products sold under the names “Silicone Fluid SS 4230” and “SS 4267” by General Electric, which are silicones with a dimethyl/trimethylsiloxane structure.

Mention may also be made of trimethylsiloxysilicate resins sold, for example, under the names X22-4914, X21-5034 and X21-5037 by Shin-Etsu.

The organomodified silicones which can be used in the compositions disclosed herein include, for example, silicones as defined above and comprising, in their structure, at least one organofunctional group attached via a hydrocarbonaceous radical.

In one embodiment, the organomodified silicones are chosen from polyorganosiloxanes comprising at least one group chosen from:

polyethyleneoxy and polypropyleneoxy groups optionally comprising at least one alkyl group chosen from C₆-C₂₄ alkyl groups, such as the product known as dimethicone copolyol sold by Dow Corning under the name DC 1248 and the oils SILWET L 722, L 7500, L 77 and L 711 from Union Carbide and the alkyl (Cl₂) methicone copolyol sold by Dow Corning under the name Q2 5200;

substituted and unsubstituted aminated groups, such as the products sold under the names GP 4 Silicone Fluid and GP 7100 by Genesee and the products sold under the names Q2 8220 and Dow Corning 929 or 939 by Dow Corning. The substituted aminated groups may, for example, be chosen from amino(C₁-C₄)alkyl groups and, further, for example, amino(C₁-C₄)alkylimino(C₁-C₄)alkyl groups;

quaternary ammonium groups, such as the products sold under the names ABILQUAT 3272 and ABILQUAT 3474 by Goldschmidt;

thiol groups, such as the products sold under the names “GP 72 A” and “GP 71” from Genesee;

alkoxylated groups, such as the product sold under the name “Silicone Copolymer F-755” by SWS Silicones and ABIL WAX 2428, 2434 and 2440 by Goldschmidt;

hydroxylated groups, such as the polyorganosiloxanes comprising at least one hydroxyalkyl functional group disclosed in French Patent Application No. FR-A-85 16334;

acyloxyalkyl groups, such as, the polyorganosiloxanes disclosed in Patent No. U.S. Pat. No. 4,957,732;

anionic groups of the carboxyl type, such as, the products disclosed in Patent No. EP 186 507 from Chisso Corporation, and anionic groups of alkylcarboxyl type, such as those present in the product X-22-3701 E from Shin-Etsu; 2-hydroxyalkylsulphonate; and 2-hydroxyalkyl thiosulphate, such as the products sold by Goldschmidt under the names “Abil S201” and “Abil S255”;

hydroxyacylamino groups, such as the polyorganosiloxanes disclosed in Patent Application No. EP 342 834. Mention may be made, for example, of the product Q2-8413 from Dow Corning.

Examples of organomodified silicones also include block copolymers comprising a linear polysiloxane-polyoxyalkylene block of (A-B)ₙ type, for example, of the following general formula:

([Y(R₂SiO)ₐR′₂SiYO][(CₙH₂ₙO)_{b}])_{c} (V)

wherein:

R₂ and R₁₂, which may be identical or different, are each chosen from monovalent hydrocarbonaceous radicals not comprising aliphatic unsaturation;

n is an integer ranging from 2 to 4;

a is an integer greater than or equal to 5, for example, ranging from 5 to 200 and, even further, for example, ranging from 5 to 100;

b is an integer greater than or equal to 4, for example, ranging from 4 to 200 and even, further, for example, ranging from 5 to 100;

c is an integer greater than or equal to 4, for example, ranging from 4 to 1 000 and, further, for example, ranging from 5 to 300; and

Y is a divalent organic group which is bonded to the adjacent silicon atom via a carbon-silicon bond and to a polyoxyalkylene block via an oxygen atom;

the average molecular weight of each siloxane block ranges from 400 to 10 000, that of each polyoxyalkylene block ranges from 300 to 10 000,

the siloxane blocks represent from 10% to 95% by weight of the block copolymer,

the weight-average molecular weight of the block copolymer being at least 3 000 and, for example, ranging from 5 000 to 1 000 000 and, further, for example, ranging from 10 000 to 200 000.

R₂ and R′₂, which may be identical or different, can each, for example, be chosen from alkyl radicals, such as, methyl, ethyl, propyl, butyl, pentyl, hexyl, octyl, decyl and dodecyl radicals; aryl radicals, such as, phenyl and naphthyl radicals; aralkyl radicals, such as, benzyl and phenylethyl radicals; and tolyl, xylyl and cyclohexyl radicals.

Y may, for example, be chosen from —R″—, —R″—CO—, —R″—NHCO—, —R″—NH—CO—NH—R—NHCO— and —R″—OCONH—R—NHCO—, wherein R″ is a divalent alkylene group, such as, an ethylene, propylene or butylene group, and R is chosen from a divalent alkylene group and a divalent arylene group, such as —C₆H₄—, —C₆H₄—C₆H₄—, —C₆H₄—CH₂—C₆H₄— and —C₆H₄—C(CH₃)₂—C₆H₄— groups.

Further, for example, Y can be a divalent alkylene radical, for example, a —CH₂—CH₂—CH₂— radical or a C₄H₈ radical.

The preparation of the block copolymers for use in the compositions disclosed herein are disclosed in European Patent Application No. EP 0 492 657 A1.

Other examples of organomodified silicones include silicone polyurethanes, such as those disclosed in Patent Application Nos. EP 0 751 162, EP 0 619 111 and EP 1 025 833.

The organomodified silicones may also be chosen, for example, from organomodified silicones comprising a polysiloxane portion and a portion comprising at least one non-silicone organic chain, one of the two portions constituting the main chain of the polymer and the other being grafted to the main chain. These polymers are, for example, disclosed in Patent Application Nos. EP-A-412 704, EP-A-412 707, EP-A-640 105 and WO 95/00578, EP-A-582 152 and WO 93/23009 and Patent Nos. U.S. Pat. No. 4,693,935, U.S. Pat. No. 4,728,571 and U.S. Pat. No. 4,972,037. These polymers may, for example, be anionic or nonionic.

Such polymers are, for example, the copolymers capable of being obtained by radical polymerization from a mixture of monomers comprising:

a) 50 to 90% by weight of tert-butyl acrylate;

b) 0 to 40% by weight of acrylic acid; and

c) 5 to 40% by weight of silicone macromer of formula:

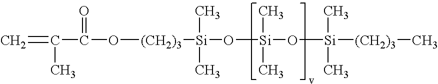

wherein v is a number ranging from 5 to 700; the percentages by weight being calculated relative to the total weight of the monomers.

The grafted silicone polymers may, for example, be chosen from polydimethylsiloxanes (PDMSs) to which mixed polymer units of the poly((meth)acrylic acid) type and of the poly(alkyl (for example, C₁-C₄) (meth)acrylate) type are grafted via a connecting link, for example, of thiopropylene type, and polydimethylsiloxanes (PDMSS) to which polymer units of the poly(isobutyl (meth)acrylate) type are grafted via a connecting link of thiopropylene type.

The at least one silicone may be provided in a form chosen from emulsions, nanoemulsions and microemulsions.

Other examples of the polyorganosiloxanes for use in the compositions disclosed herein may be chosen from:

silicones chosen from the family of the polydimethylsiloxanes comprising trimethylsilyl end groups, such as the oils of the DC 200 series from Dow Corning, for example, that with a viscosity of 60 000 cSt, oils of the SILBIONE 70047 and 47 series and, further, for example, the oil SILBIONE 70 047 V 500 000 sold by Rhodia Chimie, and the silicone oil AK 300000 from Wacker, polydimethylsiloxanes comprising dimethylsilanol end groups, such as dimethiconols, and polyalkylarylsiloxanes, such as the oil SILBIONE 70641 V 200 sold by Rhône-Poulenc;

the organopolysiloxane resin sold under the name Dow Corning 593; and

polysiloxanes comprising at least one aminated group, such as amodimethicones and trimethylsilylamodimethicones.

As used herein, the at least one silicone may, for example, be present in an amount ranging from 0.001% to 0.15% by weight, further, for example, from 0.01% to 10% by weight, and even further, for example, from 0.1 to 5% by weight, relative to the total weight of the composition.

In one embodiment, the weight ratio of the at least one amphoteric surfactant to the at least one silicone can range, for example, from 0.1:1 to 15:1 and, further, for example, ranges from 0.5:1 to 10:1.

The compositions disclosed herein may further comprise at least one additional surface-active agent which may be present, for example, in an amount ranging from 0.1% to 40% by weight, further, for example, from 3% to 30% by weight, and even further, for example, from 5% to 20% by weight, relative to the total weight of the composition.

The at least one additional surface-active agent may, for example, be chosen from anionic, amphoteric and non-ionic surface-active agents.

The at least one additional surface-active agent which is suitable for use in the composition disclosed herein may, for example, be chosen from the following:

(i) Anionic Surface-Active Agent(s):

Their nature does not assume a really critical character within the context of the embodiments disclosed herein.

For example, the anionic surface-active agents which may be used in the compositions disclosed herein may be chosen from at least one of salts, for example, alkali metal salts, such as sodium salts; ammonium salts; amine salts; aminoalcohol salts; and magnesium salts of the following compounds: alkyl sulphates, alkyl ether sulphates, alkylamido ether sulphates, alkylarylpolyether sulphates, monoglyceride sulphates; alkylsulphonates, alkyl phosphates, alkylamidesulphonates, alkylarylsulphonates, x-olefinsulphonates, paraffinsulphonates; alkyl sulphosuccinates, alkyl ether sulphosuccinates, alkylamidesulphosuccinates; alkyl sulphosuccinamates; alkyl sulphoacetates; alkyl ether phosphates; acylsarcosinates; acylisethionates and N-acyltaurates, wherein the alkyl and acyl radical of all these different compounds, for example, comprise from 8 to 24 carbon atoms, and the aryl radicals may, for example, be chosen from a phenyl group and a benzyl group.

The anionic surface-active agents may also, for example, be chosen from salts of fatty acids, such as the salts of oleic, ricinoleic, palmitic and stearic acids, the acids of coconut oil and of hydrogenated coconut oil, and acyllactylates wherein the acyl radical comprises from 8 to 20 carbon atoms. The anionic surface-active agents may also be chosen from weakly anionic surfactants, such as alkyl-D-galactosideuronic acids and salts thereof, as well as polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and salts thereof, for example, those comprising from 2 to 50 ethylene oxide groups and mixtures thereof.

The anionic surface active-agents may, for example, be chosen from at least of alkyl sulphate salts and alkyl ether sulphate salts.

(ii) Non-Ionic Surface-Active Agent(s):

The non-ionic surface-active agents which may be used in the compositions disclosed herein may be chosen from compounds which are well known per se (see, for example, “Handbook of Surfactants” by M. R. Porter, published by Blackie & Son (Glasgow and London), 1991, pp. 116-178) and, in the context of the embodiments disclosed herein, their nature does not assume any critical character. The non-ionic surface-active agents may thus be chosen, for example, from fatty alcohols, α-diols, alkylphenols and polyethoxylated, polypropoxylated and polyglycerolated fatty acids, comprising at least one fatty chain comprising, for example, from 8 to 18 carbon atoms, it being possible for the number of ethylene oxide or propylene oxide groups to range, for example, from 2 to 50 and it being possible for the number of glycerol groups to range, for example, from 2 to 30.

The non-ionic surface-active agents may also, for example, be chosen from copolymers of ethylene and propylene oxide and the condensates of ethylene and propylene oxide with fatty alcohols; polyethoxylated fatty amides, for example, comprising from 2 to 30 mol of ethylene oxide, polyglycerolated fatty amides on average comprising from 1 to 5 glycerol groups and, for example, from 1.5 to 4 glycerol groups; oxyethylenated esters of sorbitan fatty acids comprising from 2 to 30 mol of ethylene oxide; sucrose esters of fatty acids, polyethylene glycol esters of fatty acids, alkylpolyglycosides, N-alkylglucamine derivatives, and amine oxides, such as oxides of (C₁₀-C₁₄)alkylamines and N-acylaminopropylmorpholine oxides. It should be noted that alkylpolyglycosides are non-ionic surfactants which can, for example, be used in the compositions disclosed herein.

(iii) Amphoteric Surface-Active Agent(s):

The amphoteric surface-active agents, the nature of which does not assume any critical character in the context of the embodiments disclosed herein, may, for example, be chosen from derivatives of aliphatic secondary and tertiary amines wherein the aliphatic radical is chosen from linear and branched chains comprising from 8 to 22 carbon atoms and comprising at least one water-solubilizing anionic group (for example, the at least one water-solubilizing anionic group can be chosen from carboxylate, sulphonate, sulphate, phosphate and phosphonate groups); the amphoteric surface-active agents may also be chosen from (C₈-C₂₀)alkyl betaines, sulphobetaines, (C₈-C₂₀)alkyl amido(C₁-C₆)alkyl betaines and (C₈-C₂₀)alkyl amido(C₁-C₆)alkyl sulphobetaines.

For example, among the amine derivatives, the products sold under the name MIRANOL, as disclosed in Patent Nos. U.S. Pat. No. 2,528,378 and U.S. Pat. No. 2,781,354 and with the formulae (2) and (3) may be used in the compositions disclosed herein:

R₂—CONHCH₂CH₂—N(R₃)(R₄)(CH₂COO—) (2)

wherein:

R₂ is chosen from alkyl radicals derived from an acid R₂—COOH present in hydrolysed coconut oil and heptyl, nonyl and undecyl radicals;

R₃ is a β-hydroxyethyl group; and

R₄ is a carboxymethyl group; and

R₅—CONHCH₂CH₂—N(B)(A) (3)

wherein:

B is chosen from —CH₂CH₂OX′ groups; A is chosen from —(CH₂)_{z}—Y′ groups, wherein z=1 or 2;

X′ is chosen from a —CH₂CH₂—COOH group and a hydrogen atom;

Y′ is chosen from a —COOH radical and a —CH₂—CHOH—SO₃H radical; and

R₅ is chosen from alkyl radicals of a carboxylic acid present in hydrolysed linseed oil and coconut oil; and alkyl radicals, for example, a C₇, C₉, C₁₁ and C₁₃ alkyl radical; and a C₁₇ alkyl radical and the iso form thereof and an unsaturated C₁₇ radical.

These compounds are classified in the CTFA dictionary, 7th Edition, 1997, under the names Disodium Cocoamphodiacetate, Disodium Lauroamphodiacetate, Disodium Capryloamphodiacetate, Disodium Caproamphodiacetate, Disodium Cocoamphodipropionate, Disodium Lauroamphodipropionate, Disodium Caproamphodipropionate, Disodium Capryloamphodipropionate, Lauroamphodipropionic Acid and Cocoamphodipropionic Acid.

For example, disodium cocoamphodiacetate sold under the trade name Miranol® C2M concentrate by Rhodia Chimie may be used.

The compositions disclosed herein can, for example, comprise mixtures of surface-active agents and, for example, mixtures of anionic surface-active agents and, for example, mixtures of anionic surface-active agents and of amphoteric and/or non-ionic surface-active agents.

For example, the anionic surface-active agents, may, for example, be chosen from at least one of sodium, triethanolamine and ammonium (C₁₂-C₁₄)alkyl sulphates, sodium, triethanolamine and ammonium (C₁₂-C₁₄)alkyl ether sulphates oxyethylenated with 2.2 mol of ethylene oxide, sodium cocoyl isethionate and sodium α-(C₁₄-C₁₆)olefinsulphonate.

The anionic surface-active agents can, for example, be present in an amount ranging from 1 to 30% by weight, further, for example, from 3 to 20% by weight, relative to the total weight of the composition.

The amphoteric surface-active agents (other than the at least one amphoteric surfactant chosen from alkylamphohydroxyalkylsulphonate compounds and salts thereof) or non-ionic surface-active agents can, for example, be present in an amount ranging from 0.5 to 15% by weight, further, for example, from 1 to 8% by weight, relative to the total weight of the composition.

The amounts and the qualities of the at least one additional surface-active agent is that sufficient to confer a satisfactory foaming and/or detergent power on the final composition.

In the compositions disclosed herein, the combined surfactants (i.e., the at least one additional surface-active agent and the at least one amphoteric surfactant) may be present in an amount ranging from 4 to 50% by weight, further, for example, from 6 to 35% by weight, and, even further, for example, from 8 to 25% by weight relative to the total weight of the composition.

The compositions disclosed herein may also comprise at least one additive chosen from thickeners, fragrances, pearlescent agents, preservatives, sunscreens, cationic surfactants, anionic polymers, non-ionic polymers, amphoteric polymers, cationic polymers, proteins, protein hydrolysates, ceramides, pseudoceramides, fatty acids with at least one chain chosen from linear and branched C₁₆-C₄₀ chains, such as 18-methyleicosanoic acid, hydroxy acids, vitamins, provitamins, such as panthenol, vegetable oils, mineral oils and synthetic oils, antidandruff agents and any other additive conventionally used in the cosmetics field which does not affect the stability and the properties of the compositions disclosed herein.

The at least one additive optionally can be present in the composition disclosed herein in an amount ranging from 0.001 to 50% by weight, relative to the total weight of the composition. The amount of each additive desired is easily determined by a person skilled in the art according to its nature and its function.

In one embodiment, the compositions disclosed herein can further comprise cationic polymers.

The cationic polymers which can be used in the composition disclosed herein may be chosen, for example, from all those already known per se as improving the cosmetic properties of hair treated with detergent compositions, for example, those disclosed in Patent Application No. EP-A-0 337 354 and in French Patent Application Nos. FR-A-2 270 846, 2 383 660, 2 598 611, 2 470 596 and 2 519 863.

As used herein, the term “cationic polymer” means any polymer comprising at least one group chosen from cationic groups and groups which can be ionized to cationic groups.

Further, for example, the cationic polymers which can be used in the compositions disclosed herein may be chosen from quaternary derivatives of cellulose ether, such as the products sold under the name “JR 400” by Amerchol, cyclopolymers, for example, diallyldimethylammonium salt homopolymers and copolymers of diallyldimethylammonium salt and of acrylamide, for example, chlorides, sold under the names “Merquat 100”, “Merquat 550” and “Merquat S” by Ondeo, and cationic non-cellulose polysaccharides, such as guar gums modified by 2,3-epoxypropyl-trimethylammonium chloride, sold, for example, under the name “Jaguar C13S” by Rhodia Chimie.

For example, the cationic polymers may be chosen from polymers comprising at least one repeat unit corresponding to the formula:

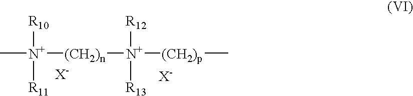

wherein R₁₀, R₁₁, R₁₂ and R₁₃, which may be identical or different, are each chosen from alkyl and hydroxyalkyl radicals comprising from 1 to 4 carbon atoms, n and p, which may be identical or different, are each integers ranging from 2 to 20, and X⁻ is chosen from anions derived from an inorganic or organic acid.

In one embodiment, the cationic polymers are present in an amount ranging, for example, from 0.001% to 10% by weight, further, for example, from 0.005% to 5% by weight, and even further, for example, from 0.01% to 3% by weight, relative to the total weight of the composition.

The cosmetically acceptable medium can comprise solely water or a mixture of water and at least one cosmetically acceptable solvent chosen for example, from lower C₁-C₄ alcohols, for example, ethanol, isopropanol, tert-butanol and n-butanol, alkylene glycols, for example propylene glycol, and glycol ethers.

The composition disclosed herein can comprise, for example, from 50 to 95% by weight of water, relative to the total weight of the composition.

In another embodiment, the compositions disclosed herein exhibit a final pH ranging from 3 to 10. This pH may range, for example, from 4 to 8. The pH may be conventionally adjusted to the desired value by addition of a base (organic or inorganic) to the composition chosen, for example, from aqueous ammonia and primary, secondary and tertiary (poly)amine, such as monoethanolamine, diethanolamine, triethanolamine, isopropanolamine and 1,3-propanediamine, or alternatively by addition of an acid chosen, for example, from a carboxylic acid, such as, citric acid.

The compositions disclosed herein may further comprise, in some embodiments, viscosity-regulating agents, such as electrolytes, or thickening agents. For example, the viscosity regulating agents may be chosen from sodium chloride, scleroglucans, xanthan gums, fatty acid alkanolamides, alkyl ether carboxylic acid alkanolamides optionally oxyethylenated with up to 5 mol of ethylene oxide, such as the product sold under the name “Aminol A15” by Chem Y, crosslinked poly(acrylic acid)s and crosslinked acrylic acid/C₁₀-C₃₀ alkyl acrylate copolymers. These viscosity-regulating agents are used in the compositions disclosed herein in amounts which can range up to 10% by weight, relative to the total weight of the composition.

The compositions disclosed herein can also comprise up to 5% by weight, of pearlescent and/or opacifying agents well known in the state of the art. The pearlescent and/or opacifying agents may be chosen, for example, from coated and non-coated titanium oxides, sodium and magnesium palmitates, sodium and magnesium stearates and hydroxystearates, acylated derivatives comprising at least one fatty chain, such as ethylene glycol and polyethylene glycol monostearates and distearates, ethers with at least one fatty chain (for example C₈-C₃₀ fatty chains), such as, distearyl ether and 1-(hexadecyloxy)-2-octadecanol, and cyclodextrins and fatty alcohols, such as cetyl, stearyl and behenyl alcohols.

The compositions disclosed herein may also comprise foam synergists. The foam synergists can, for example, be chosen from C₁₀-C₁₈ 1,2-alkanediols and fatty alkanolamides derived from monoethanolamine and from diethanolamine.

The compositions disclosed herein may be used for washing and/or treating a keratinous substance such as hair, skin, eyelashes, eyebrows, nails, lips or scalp and, for example, the hair.

For example, the compositions may be provided in a form chosen from shampoos, shower gels and foam baths.

The compositions disclosed herein may also be provided in a form chosen from rinse-out and leave-in conditioners; compositions for perming; compositions for hair-straightening; dyeing and bleaching compositions; and rinse-out compositions, to be applied before or after dyeing, bleaching, perming or hair straightening or between the two stages of a perming or of hair straightening.

The compositions disclosed herein may also be provided in the form of make-up removers.

The compositions disclosed herein may be provided in a form chosen from gels, milks, creams, emulsions, thickened lotions, and foams and can be used for the skin, scalp, nails, eyelashes, lips and, for example, the hair.

These compositions may be foaming compositions and the foaming power of the compositions disclosed herein, characterized by a foam height, may be greater than 75 mm, further, for example, greater than 100 mm, measured according to the modified Ross-Miles method (NF T 73-404/SO696).

The modifications to the method are as follows:

The measurement was carried out at a temperature of 22° C. with water purified by osmosis. The concentration of the solution was 2 g/l. The height of the drop was 1 m. The amount of composition which dropped was 200 ml. The 200 ml of composition fell into a measuring cylinder having a diameter of 50 mm and comprised 50 ml of the composition to be tested. The measurement was made 5 minutes after the composition had finished being run in.

Further disclosed herein is a process for treating a keratinous substance, such as skin or hair, comprising applying, to the keratinous substance, a cosmetic composition as defined above and optionally rinsing the keratin substance, for example, with water.

This process thus makes it possible to treat, care for, wash and/or remove make-up from the skin, hair or any other keratinous substance.

In everything which follows or precedes, the percentages expressed are by weight.

The embodiments disclosed herein will now be more fully illustrated using the following examples, which should not be regarded as limiting it to the embodiments described. In the examples, AM means active material.

### EXAMPLE 1

Two shampoo compositions were prepared, one in accordance with the present disclosure (composition A) and the other comparative (composition B):

| | | Invention | Comparative |
|---|---|---|---|
| Composition | | (A) | (B) |
| Sodium cocoylamphohydroxypropyl- | | 12 g | — |
| sulphonate, as an aqueous solution | | A.M. | |
| comprising 32% of active material, | | | |
| provided under the name Miranol CSE by | | | |
| Rhodia | | | |
| Cocoamidopropyl betaine, as an aqueous | | — | 12 g |
| solution comprising 30% of active material | | | A.M. |
| Polydimethylsiloxane with a viscosity of | | 1 g | 1 g |
| 500 000 cSt, sold under the name Mirasil | | | |
| DM 500.000 by Rhodia | | | |
| 1-(Hexadecyloxy)-2-octadecanol/ | | 2.5 g | 2.5 g |
| cetyl alcohol mixture | | | |
| Monoisopropanolamide of coconut acids | | 2 g | 2 g |
| Preservatives | | q.s. | q.s. |
| Fragrance | | q.s. | q.s. |
| Citric acid or sodium | | | |
| hydroxide | q.s. for | pH 7 | pH 7 |
| Demineralized water | q.s. for | 100 g | 100 g |

Shampooing was carried out by applying approximately 6 g of the composition A to a half-head of natural hair wetted beforehand. The shampoo was made to foam and was then rinsed copiously with water. The hair was dried with a hairdryer.

The same procedure as above was carried out with the comparative composition B on the other half-head.

The experts compared the half-heads in pairs.

A panel of experts evaluated the appearance of the dried hair and recorded a greater ease of disentangling and a greater smoothness to the touch with the composition in accordance with the present disclosure.

The hair treated with composition A was significantly smoother and disentangled more easily than hair treated with composition B.

### EXAMPLES 2 AND 3

The following shampoo compositions were prepared:

| Composition | | Example 2 | Example 3 |
|---|---|---|---|
| Sodium lauryl ether sulphate (70/30 | | 12 g A.M. | 15.5 g A.M. |
| C₁₂/C₁₄) comprising 2.2 mol of ethylene | | | |
| oxide | | | |
| Sodium cocoylamphohydroxypropyl- | | 1.8 g A.M. | 2.2 g A.M |
| sulphonate, as an aqueous solution | | | |
| comprising 32% of active material, | | | |
| provided under the name Miranol CSE by | | | |
| Rhodia | | | |
| Hydroxyethylcellulose crosslinked by | | 0.4 g | — |
| epichlorohydrin and quaternized by | | | |
| trimethylamine, sold under the name | | | |
| JR 400 by Amerchol | | | |
| Guar gum modified by 2,3-epoxy- | | — | 0.1 g |
| propyltrimethylammonium chloride, sold | | | |
| under the name Jaguar C13 S by Rhodia | | | |
| Polydimethylsiloxane with a viscosity of | | 1 g | — |
| 500 000 cSt, sold under the name Mirasil | | | |
| DM 500 000 by Rhodia | | | |
| Polydimethylsiloxane with a viscosity of | | — | 2.7 g |
| 300 000 cSt, sold under the name DC 200 | | | |
| Fluid 300 000 by Dow Corning | | | |
| 1-(Hexadecyloxy)-2-octadecanol/ | | 2.5 g | — |
| cetyl alcohol mixture | | | |
| Distearyl ether | | — | 1.5 g |
| Behenyl alcohol | | — | 1.5 g |
| Oxyethylenated sorbitan monolaurate | | 8 g | — |
| comprising 4 mol of ethylene oxide, sold | | | |
| under the name Tween 21 by Uniqema | | | |
| Monoisopropanolamide of coconut acids | | 0.1 g | 1 g |
| Crosslinked poly(acrylic acid) | | 0.2 g | 0.2 g |
| Preservatives | | q.s. | q.s. |
| Fragrance | | q.s. | q.s. |
| Citric acid | q.s. | pH 5.3 | pH 7 |
| Demineralized water | q.s. for | 100 g | 100 g |

The hair washed with these compositions easily disentangled, was smooth (to the touch and visually) and was manageable (supple).

### EXAMPLE 4

The following shampoo composition was prepared:

| Sodium lauryl ether sulphate (70/30 | | 15.4 g A.M. |
|---|---|---|
| C₁₂/C₁₄) comprising 2.2 mol of ethylene | | |
| oxide | | |
| Sodium cocoylamphohydroxypropyl- | | 2.4 g A.M. |
| sulphonate, as an aqueous solution | | |
| comprising 32% of active material, | | |
| provided under the name Miranol CSE by | | |
| Rhodia | | |
| Dimethyl diallyl ammonium chloride | | 0.48 g AM |
| homopolymer, in 40% AM aqueous | | |
| solution, provided under the name | | |
| Merquat 100 by NALCO | | |
| Polydimethylsiloxane with groups | | 1.2 g AM |
| aminoéthyl iminobutyl and α, ω silanols in | | |
| 60% AM aqueous cationic emulsion, | | |
| provided under the name DC2-8299 by | | |
| DOW CORNING | | |
| Distearyl ether | | 1.5 g |
| Behenyl alcohol | | 1.5 g |
| Crosslinked poly(acrylic acid) | | 0.4 g |
| Preservatives | | q.s. |
| Fragrance | | q.s. |
| Citric acid | q.s. | pH 5.1 |
| Demineralized water | q.s. for | 100 g |

What is claimed is:

1. A cosmetic composition comprising, in a cosmetically acceptable medium,
at least one amphoteric surfactant chosen from alkylamphohydroxyalkylsulphonate compounds and salts thereof and
at least one silicone chosen from non-organomodified silicones with a viscosity ranging from 500 mm²/s (500 centistokes) to 1 000 000 mm²/s (1 000 000 centistokes) and organomodified silicones.

2. The composition according to claim 1, wherein the at least one amphoteric surfactant is chosen from alkylamphohydroxyalkylsulphonate compounds and salts thereof of the following formula (I): 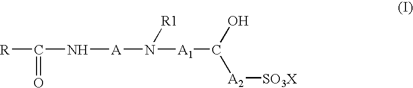
wherein:
R is chosen from saturated and unsaturated and linear and branched hydrocarbonaceous radicals comprising from 5 to 29 carbon atoms;
R1 is chosen from C₁-C₄ hydroxyalkyl radicals;
A, A₁ and A₂, which may be identical or different, are each chosen from linear and branched, divalent, C₁-C₁₀ radicals; and
X is chosen from a hydrogen atom and inorganic and organic cations.

3. The composition according to claim 2, wherein, R1 is a hydroxyethyl radical.

4. The composition according to claim 2, wherein, A, A₁ and A₂, which may be identical or different, are each chosen from C₁-C₃ alkylene radicals.

5. The composition according to claim 2, wherein, R is chosen from alkyl and mono- and polyunsaturated alkenyl radicals comprising from 5 to 29 carbon atoms.

6. The composition according to claim 5, wherein, R is chosen from alkyl radicals and mono- and polyunsaturated alkenyl radicals comprising from 7 to 22 carbon atoms.

7. The composition according to claim 6, wherein, R is chosen from alkyl radicals and mono- and polyunsaturated alkenyl radicals comprising from 9 to 17 carbon atoms.

8. The composition according to claim 2, wherein, A and A₂ are each a —CH₂CH₂— radical.

9. The composition according to claim 2, wherein, A₁ is a —CH₂— radical.

10. The composition according to claim 2, wherein, the inorganic and organic cations are chosen from alkali metals, alkaline earth metals, a NH₄⁺ ion, and ammonium ions resulting from basic amino acids and from aminoalcohols.

11. The composition according to claim 10, wherein the alkali metals are chosen from Na⁺ and K⁺ ions.

12. The composition according to claim 10, wherein the alkaline earth metals are chosen from Mg²⁺ and Ca²⁺ ions.

13. The composition according to claim 1, wherein the at least one amphoteric surfactant is chosen from salts of cocoylamphohydroxypropylsulphonate and salts of palmitoylamphohydroxypropylsulphonate.

14. The composition according to claim 1, wherein the at least one amphoteric surfactant is present in an amount ranging from 0.1% to 30% by weight, relative to the total weight of the composition.

15. The composition according to claim 14, wherein the at least one amphoteric surfactant is present in an amount ranging from 1% to 20% by weight, relative to the total weight of the composition.

16. The composition according to claim 1, wherein the at least one silicone is provided in a form chosen from oils, waxes, resins, and gums.

17. The composition according to claim 1, wherein the non-organomodified silicones are polysiloxanes chosen from at least one of polydialkylsiloxanes, polydiarylsiloxanes, polyalkylarylsiloxanes, silicone gums, and silicone resins.

18. The composition according to claim 17, wherein:
(a) the polydialkylsiloxanes are chosen from:
polydimethylsiloxanes comprising trimethylsilyl end groups;
polydimethylsiloxanes comprising dimethylsilanol end groups; and
poly(C₁-C₂₀)alkylsiloxanes;
(b) the polyalkylarylsiloxanes are chosen from:
linear and branched polydimethylmethylphenylsiloxanes and polydimethyldiphenylsiloxanes;
(c) the silicone gums are chosen from polydiorganosiloxanes having number-average molecular masses ranging from 200,000 to 1,000,000, wherein the polydiorganosiloxanes are used alone or as a mixture in a solvent;
(d) the silicone resins are chosen from silicone resins comprising at least one unit chosen from R₃SiO_{1/2}, R₂SiO_{2/2}, RSiO_{3/2} and SiO_{4/2}, wherein R is chosen from hydrocarbonaceous groups comprising from 1 to 16 carbon atoms and a phenyl group.

19. The composition according to claim 18, wherein the silicone gums are chosen from at least one of the following:
polydimethylsiloxane;
polydimethylsiloxane/methylvinylsiloxane;
polydimethylsiloxane/diphenylsiloxane;
polyd imethylsiloxane/phenylmethylsiloxane;
polydimethylsiloxane/diphenylsiloxane/methylvinylsiloxane; and the following mixtures:
mixtures formed from a polydimethylsiloxane hydroxylated at the chain end and from a cyclic polydimethylsiloxane;
mixtures formed from a polydimethylsiloxane gum and from a cyclic silicone; and
mixtures of polydimethylsiloxanes with different viscosities.

20. The composition according to claim 1, wherein the organomodified silicones are chosen from polyorganosiloxanes comprising at least one group chosen from:
a) polyethyleneoxy and polypropyleneoxy groups;
b) substituted and unsubstituted aminated groups;
c) thiol groups;
d) alkoxylated groups;
e) hydroxyalkyl groups;
f) acyloxyalkyl groups;
g) alkylcarboxyl groups;
h) 2-hydroxyalkylsulphonate groups;
i) 2-hydroxyalkyl thiosulphate groups;
j) hydroxyacylamino groups; and
k) quaternary ammonium groups.

21. The composition according to claim 1, wherein the at least one silicone is chosen from polyalkylsiloxanes comprising trimethylsilyl end groups, polyalkylsiloxanes comprising dimethylsilanol end groups, polyalkylarylsiloxanes, organopolysiloxane resins and polysiloxanes comprising aminated groups.

22. The composition according to claim 1, wherein the at least one silicone is chosen from polyorganosiloxanes which are insoluble in the composition.

23. The composition according to claim 1, wherein the at least one silicone is present in an amount ranging from 0.001% to 15% by weight, relative to the total weight of the composition.

24. The composition according to claim 23, wherein the at least one silicone is present in an amount ranging from 0.01% to 10% by weight, relative to the total weight of the composition.

25. The composition according to claim 24, wherein the at least one silicone is present in an amount ranging from 0.1% to 5% by weight, relative to the total weight of the composition.

26. The composition according to claim 1, wherein the weight ratio of the at least one amphoteric surfactant to the at least one silicone ranges from 0.1:1 to 15:1.

27. The composition according to claim 26, wherein the weight ratio of the at least one amphoteric surfactant to the at least one silicone ranges from 0.5:1 to 10:1.

28. The composition according to claim 1, further comprising at least one additional surface-active agent chosen from anionic, cationic, nonionic and amphoteric surfactants.

29. The composition according to claim 28, wherein the at least one additional surface-active agent is present in an amount ranging from 0.1% to 40% by weight, relative to the total weight of the composition.

30. The composition according to claim 29, wherein the at least one additional surface-active agent is present in an amount ranging from 3% to 30% by weight, relative to the total weight of the composition.

31. The composition according to claim 30, wherein the at least one additional surface-active agent is present in an amount ranging from 5% to 20% by weight, relative to the total weight of the composition.

32. The composition according to claim 1, further comprising at least one additive chosen from thickeners, fragrances, pearlescent agents, opacifying agents, preservatives, sunscreens, anionic polymers, non-ionic polymers, amphoteric polymers, cationic polymers, proteins, protein hydrolysates, ceramides, pseudoceramides, fatty acids comprising at least one chain chosen from linear and branched C₁₆-C₄₀ chains, hydroxy acids, vitamins, provitamins, vegetable oils, mineral oils, synthetic oils and antidandruff agents.

33. The composition according to claim 32, wherein the fatty acids comprising at least one chain chosen from linear and branched C₁₆-C₄₀ chains are 18-methyleicosanoic acids.

34. The composition according to claim 32, wherein the provitamins are panthenols.

35. The composition according to claim 32, wherein the cationic polymers are chosen from:
diallyidimethylammonium salt homopolymers and copolymers of diallyldimethylammonium salt and of acrylamide;
quaternary derivatives of cellulose ether;
cationic non-cellulose polysaccharides; and
polymers comprising at least one repeat unit corresponding to the formula: 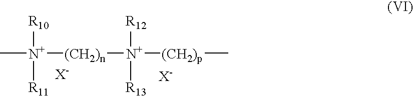
wherein R₁₀, R₁₁, R₁₂ and R₁₃, which may be identical or different, are each chosen from alkyl and hydroxyalkyl radicals comprising from 1 to 4 carbon atoms, n and p, which may be identical or different, are each integers ranging from 2 to 20 and X⁻ is chosen from anions derived from inorganic or organic acids.

36. The composition according to claim 35, wherein the cationic non-cellulose polysaccharides are chosen from cationic guar gums.

37. The composition according to claim 32, wherein the cationic polymers are present in an amount ranging from 0.001% to 10% by weight, relative to the total weight of the composition.

38. The composition according to claim 37, wherein the cationic polymers are present in an amount ranging from 0.005% to 5% by weight, relative to the total weight of the composition.

39. The composition according to claim 32, wherein the pearlescent agents and opacifying agents are chosen from coated and non-coated titanium oxides, sodium and magnesium palmitates, sodium and magnesium stearates and hydroxystearates, acylated derivatives comprising at least one fatty chain, ethers comprising at least one fatty chain, cyclodextrins and fatty alcohols.

40. The composition according to claim 39, wherein the acylated derivatives comprising at least one fatty chain are chosen from ethylene glycol, polyethylene glycol monostearates, and polyethylene glycol distearates.

41. The composition according to claim 39, wherein the ethers comprise at least one fatty chain chosen from C₈-C₃₀ fatty chains.

42. The composition according to claim 39, wherein the ethers, comprising at least one fatty chain, are chosen from distearyl ether and 1-(hexadecyloxy)-2-octadecanol.

43. The composition according to claim 39, wherein the fatty alcohols are chosen from cetyl, stearyl and behenyl alcohols.

44. The composition according to claim 1, wherein the composition is provided in a form chosen from shampoos; washing compositions for the skin; rinse-out and leave-in conditioners; compositions for perming; compositions for hair-straightening; dyeing and bleaching compositions; and rinse-out compositions, to be applied before or after dyeing, bleaching, perming or hair straightening or between the two stages of perming or hair straightening.

45. The composition according to claim 1, wherein the final pH of the composition ranges from 3 to 10.

46. The composition according to claim 45, wherein the final pH of the composition ranges from 4 to 8.

47. A method for washing a keratinous substance comprising applying to the keratinous substance a composition comprising, in a cosmetically acceptable medium,
at least one amphoteric surfactant chosen from alkylamphohydroxyalkylsulphonate compounds and salts thereof and
at least one silicone chosen from non-organomodified silicones with a viscosity ranging from 500 mm²/s (500 centistokes) to 1 000 000 mm/s (1 000 000 centistokes) and organomodified silicones.

48. The method according to claim 47, wherein the keratinous substance is hair.

49. A method for providing at least one of the following to hair: ease of disentangling, smoothness, volume, lightness, softness, suppleness and manageability comprising applying to the hair a composition comprising, in a cosmetically acceptable medium,
at least one amphoteric surfactant chosen from alkylamphohydroxyalkylsulphonate compounds and salts thereof and
at least one silicone chosen from non-organomodified silicones with a viscosity ranging from 500 mm²/s (500 centistokes) to 1 000 000 mm²/s (1 000 000 centistokes) and organomodified silicones.

50. A composition for providing at least one of the following to hair: ease of disentangling, smoothness, volume, lightness, softness, suppleness and manageability comprising, in a cosmetically acceptable medium,
at least one amphoteric surfactant chosen from alkylamphohydroxyalkylsulphonate compounds and salts thereof and
at least one silicone chosen from non-organomodified silicones with a viscosity ranging from 500 mm²/s (500 centistokes) to 1 000 000 mm²/s (1 000 000 centistokes) and organomodified silicones;
wherein the at least one amphoteric surfactant and the at least one silicone are present in a combined amount effective for providing at least one of the following to hair: ease of disentangling, smoothness, volume, lightness, softness, suppleness and manageability.

51. A process for treating a keratinous substance comprising:
applying to the keratinous substance, a cosmetic composition comprising in a cosmetically acceptable medium,
at least one amphoteric surfactant chosen from alkylamphohydroxyalkylsulphonate compounds and salts thereof and
at least one silicone chosen from non-organomodified silicones with a viscosity ranging from 500 mm²/s (500 centistokes) to 1 000 000 mm²/s (1 000 000 centistokes) and organomodified silicones; and
optionally rinsing the keratinous substance.

52. The process according to claim 51, wherein the keratinous substance is hair.

53. A method of manufacturing a cosmetic composition comprising including in the composition at least one amphoteric surfactant chosen from alkylamphohydroxyalkylsulphonate compounds and salts thereof and at least one silicone chosen from non-organomodified silicones with a viscosity ranging from 500 mm²/s (500 centistokes) to 1 000 000 m²/s (1 000 000 centistokes) and organomodified silicones.
